(19) United States
(12) Patent Application Publication
(10) Pub. No.: US 2023/0373942 A1
(43) Pub. Date: Nov. 23, 2023

(54) Process for the Production of Cannabinol, Cannabinolic Acid, Cannabivarin, Cannabivarinic Acid and Related Cannabinoids

(71) Applicants: Barry A. Berkowitz, Framingham, MA (US); Anthony G. Barrett, Rio de Janeiro (BR); Daniel Elliott, Basel (CH); Philip J. Parsons, East Sussex (GB)

(72) Inventors: Barry A. Berkowitz, Framingham, MA (US); Anthony G. Barrett, Rio de Janeiro (BR); Daniel Elliott, Basel (CH); Philip J. Parsons, East Sussex (GB)

(21) Appl. No.: 18/247,573

(22) PCT Filed: Sep. 30, 2021
(86) PCT No.: PCT/US2021/052844

§ 371 Date: Mar. 31, 2023

Related U.S. Application Data
(60) Provisional application No. 63/086,653, filed on Oct. 2, 2020.

(51) Int. Cl. C07D 311/80

(52) U.S. Cl. CPC C07D 311/80

(57) ABSTRACT

A process for the preparation of diverse known and novel cannabinoids (5), which include cannabinol (CBN, 1), cannabinolic acid (CBNA, 2), cannabivarin (CBV, 3) and cannabivarinic acid (CBVA, 4) and other naturally occurring monocyclic and tricyclic cannabinoids and other analogues from simple inexpensive starting materials using a cascade sequence of allylic rearrangement, aromatization and, as appropriate, further highly selective cyclization reactions. Additionally, cannabinoids of the formula (5) and intermediates of the formula (9) form part of the invention. Use of medicaments comprising one or more of the disclosed cannabinoids alone or admixed with one or more other cannabinoids is also disclosed.

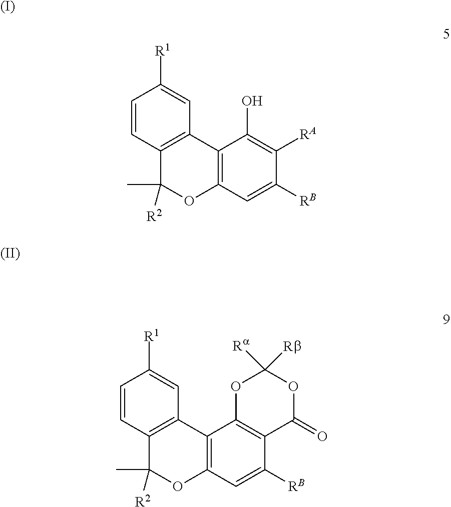

### FIELD OF THE INVENTION

The field of the invention relates to methods for the synthesis of high purity known and novel cannabinoids including but not limited to cannabinol (CBN, 1), cannabinolic acid (CBNA, 2), cannabivarin (CBV, 3) and cannabivarinic acid (CBVA, 4) and other naturally occurring cannabinoids and other synthetic analogues from simple inexpensive starting materials by construction of the aromatic core. The field of the invention additionally covers novel cannabinoids, which may be used as active compounds either alone or admixed in combination with known cannabinoids or other drugs in drug formulations for the treatment of pain, multiple sclerosis-related spasticity, nausea, anorexia, epilepsy, Alzheimer's and neurodegenerative diseases, brain injury/concussion/traumatic brain injury, stroke, cancer, reduction of inflammation and immuno-inflammation related diseases, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation among other diseases, and as anti-oxidants.

### BACKGROUND OF THE INVENTION

Cannabis sativa (“marijuana”) is a hemp plant of considerable notoriety and use. Its use as a recreational drug worldwide, has been and remains the subject of legal review in many countries of the world. There has been very considerable interest in the use of this plant and its extracts as ethnopharmaceuticals for millennia with reference even in Herodotus, (The Histories, Book IV, page 295, Penguin Books, Ltd., Middlesex (1972). The plant and its extracts have been used in medicine on account of their effects as anesthetics, spasmolytics, and hypnotic agents, immune-inflammation regulatory agents to combat the side effects of nausea following cancer chemotherapy, in the treatment of glaucoma, neuropathic pain, epilepsy, multiple sclerosis-related spasticity and pain in patients with advanced cancer, AIDS-related anorexia and pain.

There are over 60 constituent compounds that have been isolated and characterized from Cannabis sativa oil (for example see S. A. Ahmed, S. A. Ross, D. Slade, M. M. Radwan, F. Zulfiqar and M. A. ElSohly “Cannabinoid Ester Constituents from High-Potency Cannabis sativa”, Journal of Natural Products, 2008, volume 71, pages 536-542; Lewis, M. M.; Yang, Y.; Wasilewski, E.; Clarke, H. A.; Kotra, L. P., “Chemical Profiling of Medical Cannabis Extracts”, ACS Omega, 2017, volume 2, pages 6091-6103 and references therein). In addition, a considerable number of these natural products and analogs have been prepared by total synthesis from aromatic and monoterpene precursor compounds. Such total syntheses are reported (for examples see R. K. Razdan, “The Total Synthesis of Cannabinoids” in “The Total Synthesis of Natural Products”, Editor J. ApSimon, 1996, volume 4, pages 185-262, New York, N.Y.: Wiley and Sons; J. W. Huffman and J. A. H. Lainton, “Recent Developments in the Medicinal Chemistry of Cannabinoids”, Current Medicinal Chemistry, 1996, volume 3, pages 101-116; N. Itagaki, T. Sugahara and Y. Iwabuchi, “Expedient Synthesis of Potent Cannabinoid Receptor Agonist (−)-CP55,940”, Organic Letters, 2005, volume 7, pages 4181-4183; J. A. Teske and A. Deiters, “A Cyclotrimerization Route to Cannabinoids”, Organic Letters, 2008, volume 10, pages 2195-2198; S. Tchilibon and R. Mechoulam, “Synthesis of a Primary Metabolite of Cannabidiol”, Organic Letters, 2000, volume 2, pages 3301-3303; Y. Song, S. Hwang, P. Gong, D. Kim and S. Kim*, “Stereoselective Total Synthesis of (−)-Perrottetinene and Assignment of Its Absolute Configuration”, Organic Letters, 2008, volume 10, pages 269-271; Y. Kobayashi, A. Takeuchi and Y.-G. Wang, “Synthesis of Cannabidiols via Alkenylation of Cyclohexenyl Monoacetate”, Organic Letters, 2006, volume 8, pages 2699-2702; B. M. Trost and K. Dogra, “Synthesis of (−)-Δ⁹-trans-Tetrahydrocannabinol: Stereocontrol via Mo-Catalyzed Asymmetric Allylic Alkylation Reaction”, Organic Letters, 2007, volume 9, pages 861-863; L.-J. Cheng, J.-H. Xie, Y. Chen, L.-X. Wang and Q.-L. Zhou, “Enantioselective Total Synthesis of (−)-Δ⁸-THC and (−)-Δ⁹-THC via Catalytic Asymmetric Hydrogenation and S_{N}Ar Cyclization” Organic Letters, 2013, volume 15, pages 764-767; P. R. Nandaluru and G. J. Bodwell, “Multicomponent Synthesis of 6H-Dibenzo[b,d]pyran-6-ones and a Total Synthesis of Cannabinol”, Organic Letters, 2012, volume 14, pages 310-313; S. Ben-Shabat, L. O. Hanus, G. Katzavian and R. Gallily, “New Cannabidiol Derivatives: Synthesis, Binding to Cannabinoid Receptor, and Evaluation of Their Antiinflammatory Activity”, Journal of Medicinal Chemistry, 2006, volume 49, pages 1113-1117; A. Mahadevan, C. Siegel, B. R. Martin, M. E. Abood, I. Beletskaya and R. K. Razdan, “Novel Cannabinol Probes for CB1 and CB2 Cannabinoid Receptors”, Journal of Medicinal Chemistry, 2000, volume 43, pages 3778-3785; S. P. Nikas, S. O. Alapafuja, I. Papanastasiou, C. A. Paronis, V. G. Shukla, D. P. Papahatjis, A. L. Bowman, A. Halikhedkar, X. Han and A. Makriyannis, “Novel 1′,1′-Chain Substituted Hexahydrocannabinols: 9β-Hydroxy-3-(1-hexyl-cyclobut-1-yl)-hexahydrocannabinol (AM2389) a Highly Potent Cannabinoid Receptor 1 (CB1) Agonist”, Journal of Medicinal Chemistry, 2010, volume 53, pages 6996-7010; Kavarana, M. J.; Peet, R. C., “Bioenzymatic Synthesis Of THC-V, CBY And CBN and their use as Therapeutic Agents”, US Patent Application, 2017/0283837 AI; Winnicki, R.; Donsky, M.; Sun, M.; Peet, R., “Apparatus and Methods for Biosynthetic Production of Cannabinoids', U.S. Pat. No. 9,879,292 B2; Giorgi, P. D.; Liautard, V.; Pucheault, M.; Antoniotti, S. “Biomimetic Cannabinoid Synthesis Revisited: Batch and Flow All-Catalytic Synthesis of (±)-ortho-Tetrahydrocannabinols and Analogues from Natural Feedstocks”, European Journal of Organic Chemistry, 2018, pages 1307-1311; Morimoto, S.; Komatsu, K.; Taura, F.; Shoyama, Y., “Enzymological Evidence for Cannabichromenic Acid Biosynthesis”, Journal of Natural Products, 1997, volume 60, pages 854-857; Saimoto, H.; Yoshida, K.; Murakami, T.; Morimoto, M.; Sashiwa, H.; Shigemasa, Y., “Effect of Calcium Reagents on Aldol Reactions of Phenolic Enolates with Aldehydes in Alcohol”, The Journal of Organic Chemistry, 1996, volume 61, pages 6768-6769; Pollastro, F.; Caprioglio, D.; Marotta, P.; Moriello, A. S.; De Petrocellis, L.; Taglialatela-Scafati, O.; Appendino, G., “Iodine-Promoted Aromatization of p-Menthane-Type Phytocannabinoids”, Journal of Natural Products, 2018, volume 81, pages 630-633; Bastola, K. P.; Hazekamp, A.; Verpoorte, R., “Synthesis and Spectroscopic Characterization of Cannabinolic Acid”, Planta Medica, 2007, volume 73, pages 273-275).

In the last twenty years it has become apparent that the cannabinoids are in a renaissance for diverse biomedical uses. The pharmacology of the cannabinoids has been shown to be associated with a number of receptors and mechanisms including cannabinoids receptors, GPCR receptors, serotonin receptors, modulation of several voltage-gated channels (including Ca²⁺, Na⁺, and various type of K⁺ channels), ligand-gated ion channels (i.e., GABA, glycine and TRPV), Toll like receptors, opioid receptors, NMDA or excitatory amino acids receptors, catecholamine receptors, enzymes regulating endocannabinoids, and ion-transporting membranes proteins such as transient potential receptor class (TRP) channels (L. De Petrocellis, M. Nabissi, G. Santoni and A. Ligresti, “Actions and Regulation of Ionotropic Cannabinoid Receptors”, Advances in Pharmacology, 2017, volume 80, pages 249-289; P. Morales and P. H. Reggio, “An Update on Non-CB₁, Non-CB₂ Cannabinoid Related G-Protein-Coupled Receptors”, Cannabis Cannabinoid Research, 2017, volume 2, pages 265-273). Thus, it would be helpful to have a new medicament or medicaments that include one or more cannabinoids for treatment of afflictions known to be treatable by affecting or using these physiological mechanisms.

The pharmacology of the cannabinoids is directly or indirectly receptor-mediated for example, by two G protein-coupled receptors, named CB₁ and CB₂, which have 44% sequence homology in humans. The CB₁ sub-type is the most widely expressed G protein-coupled receptor in the brain in regions, for example, that control motor, emotional, cognitive, sensory responses, perception of pain, thermoregulation, as well as cardiovascular, gastrointestinal, and respiratory physiology. It is localized in the central (CNS) and peripheral nervous systems including the olfactory bulb, cortical areas, parts of the basal ganglia, thalamus, hypothalamus, cerebellar cortex, brainstem, and spinal cord. CB₁ receptors also occur in cells in the pituitary and thyroid glands, some fat, muscle and liver cells as well as the lung and kidneys. The CB₂ sub-type is expressed in immune and hematopoietic cells, osteoclasts, and osteoblasts and mediates the response of the immune system, controls inflammation, modulates inflammatory and neuropathic pain as well as bone remodeling.

The pharmacology of modulators of CB₁ and CB₂ receptors has been reviewed for example by Vemuri and Makriyannis (V. K. Vemuri and A. Makriyannis, “Medicinal Chemistry of Cannabinoids”, Clinical Pharmacology & Therapeutics, 2015, volume 97, pages 553-558). The psychoactive effects of Δ⁹-tetrahydrocannabinol (THC) as well as with its primary metabolite 11-hydroxy-Δ⁹-tetrahydrocannabinol are mediated by its partial agonism of CNS CB₁ receptors (J. van Amsterdam, T. Brunt and W. van den Brink, “The adverse health effects of synthetic cannabinoids with emphasis on psychosis-like effects”, Journal of Psychopharmacology, 2015, volume 29, pages 254-263; R. G. Pertwee, “The diverse CB₁ and CB₂ receptor pharmacology of three plant cannabinoids: Δ⁹-tetrahydrocannabinol, cannabidiol and Δ⁹-tetrahydrocannabivarin”, British Journal of Pharmacology, 2008, volume 153, pages 199-215). It is useful as an analgesic, an antiemetic agent, and for treating anorexia in patients with AIDS. Other CB₁ receptor modulators include tetrahydrocannabivarin (THCV) (weak antagonist) and cannabinol (CBN) (weak agonist) and both are modest agonists of CB₂. Both the non-psychoactive (−)-cannabidiol (CBD) and cannabidivarin (CBDV) do not interact significantly with either receptor sub-class and their modes of action are less clear (J. Fernández-Ruiz, O. Sagredo, M. R. Pazos, C. Garcia, R. Pertwee, R. Mechoulam, J. Martinez-Orgado, “Cannabidiol for neurodegenerative disorders: important new clinical applications for this phytocannabinoid?”, British Journal of Clinical Pharmacology, 2013, volume 75, pages 323-333; S. Rosenthaler, B. Pöhn, C. Kolmanz, C. N. Huu, C. Krewenka, A. Huber, B. Kranner, W.-D. Rausch and R. Moldzio, “Differences in receptor binding affinity of several phytocannabinoids do not explain their effects on neural cell cultures”, Neurotoxicology and Teratology, 2014, volume 46, pages 49-56). The combination of Δ⁹-tetrahydrocannabinol (THC) and cannabidiol (CBD) (Sativex, Nabiximols) is used to treat multiple sclerosis-related spasticity and as a potent analgesic in patients with advanced stage cancers. More recently, purified cannabidiol (CBD) was granted orphan drug status for treating epilepsy. CB₁ receptor antagonists are appetite suppressants, enhance cognition, and control addictive behavior. Selective CB₂ agonists may provide superior analgesic agents and immunomodulators that do not have the undesirable psychoactive effects associated with CNS CB₁ agonism. Δ⁹-tetrahydrocannabinol (THC) (Dronabinol) has been shown to be clinically effective either in monotherapy or in combination with ondansetron (Zofran, a 5-HT₃ antagonists) and in combination with prochlorperazine (a dopamine D₂ receptor antagonist) to treat chemotherapy-induced nausea and vomiting in cancer patients (M. B. May and A. E Glode, “Dronabinol for chemotherapy-induced nausea and vomiting unresponsive to antiemetics”, Cancer Management and Research, 2016, volume 8, pages 49-55).

Cannabinoids that are used as therapeutics are either obtained from the fractionation of Cannabis sativa oil or from total synthesis usually from aromatic and terpene starting materials. Since there are over 60 different natural products in cannabis oil, such oil fractionation requires extensive chromatographic purification to provide any individual constituent substantially pure (>99% pure) and, with so many components, makes reproducible production and storage difficult. For example, the purification of 49-tetrahydrocannabinol (THC) from other cannabis constituents but particularly from its isomer Δ⁸-tetrahydrocannabinol is inefficient and costly. In addition, since many of the cannabinoids in cannabis oil have different effects as total, partial, inverse or neutral agonists or antagonists of either or both of the CB₁ and CB₂ receptors, it is especially important that individual isolated natural products do not contain significant levels (below parts per million levels) of any other cannabinoid natural product, which has undesired biological effects and that the specifications set are efficiently reproducible. There is an added complication in that many cannabinoid natural products are obtained as oils, which are typically not possible to crystallize and which are prone to air oxidative degradation and their isolation requires the use of extensive expensive and difficult to scale chromatography and/or derivatisation (for example see B. Trawick and M. H. Owens, “Process for the Preparation of (−)-delta 9-Tetrahydrocannabinol”, WO 2009/099868 AI; E. Arslantas and U. Weigl, “Method for Obtaining Pure Tetrahydrocannabinol”, U.S. Pat. No. 7,923,558 B2; J. E. Field, J. Oudenes, B. I. Gorin, R. Orprecio, F. E. Silva e Souza, N. J. Ramjit and E.-L. Moore, “Separation of Tetrahydrocannabinols”, U.S. Pat. No. 7,321,047 B2; P. Bhatarah, K. J. Batchelor, D. McHattie and A. K. Greenwood, “Delta 9 Tetrahydrocannabinol Derivatives”, WO 2008/099183 AI; D. C. Burdick, S. J. Collier, F. Jos, B. Biolatto, B. J. Paul, H. Meckler, M. A. Helle and A. J. Habershaw, “Process for Production of Delta-9-Tetrahydrocannabinol”, U.S. Pat. No. 7,674,922 B2).

The cannabinoids cannabinol (CBN, 1), cannabinolic acid (CBNA, 2), cannabivarin (CBV, 3), cannabivarinic acid (CBVA, 4) have also been isolated and characterized from Cannabis sativa oil in variable purities. Cannabinol (CBN, 1) and cannabivarin (CBV, 3) are constituents of Cannabis sativa oil that are respectively formed on aging of cannabis in air and sunlight by the oxidative aromatization of Δ⁹-tetrahydrocannibinolic acid (THCA) or (−)-Δ⁹-trans-tetrahydrocannabinol (THC) and tetrahydrocannabivarinic acid or tetrahydrocannabivarin.

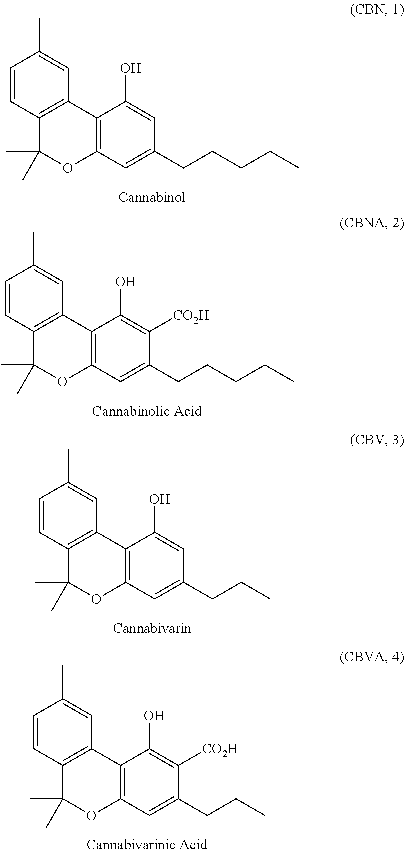

Many of the known synthetic routes to prepare cannabinoids either use expensive reagents and are uneconomic to use on a large scale or are dependent on the condensation reactions of monoterpene starting materials with derivatives of alkyl-resorcinol such as 5-n-pentyl-resorcinol (olivetol) under acidic reaction conditions, reactions that frequently give rise to side products derived from carbenium ion rearrangement reactions and/or side reactions. For example, the manufacture of Δ⁹-tetrahydrocannabinol (THC) from olivetol and monoterpenes by Brønsted or Lewis acid catalyzed condensation reactions is complicated by the co-formation of its isomer Δ⁸-tetrahydrocannabinol, amongst other impurities. Such impurities also considerably complicate and increase the cost of obtaining cannabinoid active pharmaceutical ingredients substantially pure (for examples see R. K. Razdan, “The Total Synthesis of Cannabinoids” in “The Total Synthesis of Natural Products”, Editor J. ApSimon, 1996, volume 4, pages 185-262, New York, N.Y.: Wiley and Sons; C. Steup and T. Herkenroth, “Process for Preparing Synthetic Cannabinoids”, US Patent Application 2010/0298579 AI; R. J. Kupper, “Cannabinoid Active Pharmaceutical Ingredient for Improved Dosage Forms”, WO 2006/133941 A2; J. Erler, and S. Heitner, “Method for the Preparation of Dronabinol”, U.S. Pat. No. 8,324,408 B2; A. L. Gutman, M. Etinger, I. Fedotev, R. Khanolkar, G. A. Nisnevich, B. Pertsikov, I. Rukhman and B. Tishin, “Methods for Purifying trans-(−)-Δ⁹-Tetrahydrocannabinol and trans-(+)-Δ⁹-Tetrahydrocannabinol”, U.S. Pat. No. 9,278,083 B2).

Cannabinol (CBN, 1) has been synthesized from Δ⁹-tetrahydrocannabinol (THC) by dehydrogenation with sulfur at 250° C. or using stoichiometric quantities of 2,3,5,6-tetrachloro-1,4-benzoquinone or iodine. Cannabinol (CBN, 1) acts as a partial agonist at the CB₁ receptor but has a higher affinity to CB₂ receptors. CBN has been claimed to be useful for the for treatment of neuro-behavioral disorders, insomnia, post-traumatic stress disorder, anxiety disorders such as ADHD and to be able to stimulate appetite and to be an analgesic. CBN (1) is as an activator and desensitizer of TRPA1 in a (rare) non-covalent fashion and it is an inhibitor of TRPM8 and modulator of TRPV4 cation channels. Additionally, CBN (1) may affect inflammation and has been proposed to reduce arthritis (Turner, S. E.; Williams, C. M.; Iversen, L.; Whalley, B. J., “Molecular Pharmacology of Phytocannabinoids”, Phytocannabinoids, 2017, pages 61-101; Pollastro, F.; Caprioglio, D.; Marotta, P.; Moriello, A. S.; De Petrocellis, L.; Taglialatela-Scafati, O.; Appendino, G., “Iodine-Promoted Aromatization of p-Menthane-Type Phytocannabinoids”, Journal of Natural Products, 2018, volume 81, pages 630-633; Bray, D. H.; Lap, M.; Dupetit, A. C., “Composition for the treatment of neurobehavioral disorders”, U.S. Pat. No. 9,763,991 B2; Zurier, R. B.; Burstein, S. H., “Cannabinoids, inflammation, and fibrosis”, FASEB, 2016, volume 30, pages 3682-3689; Booker, L.; Naidu, P. S.; Razdan, R. K.; Mahadevan, A.; Lichtman, A. H., “Evaluation of prevalent phytocannabinoids in the acetic acid model of visceral nociception”, Drug and Alcohol Dependence, 2009, volume 105, pages 42-47; Farrimond, J. A.; Whalley, B. J.; Williams, C. M., “Cannabinol and cannabidiol exert opposing effects on rat feeding patterns”, Psychopharmacology, 2012, volume 223, pages 117-129).

Biomedical studies of cannabivarin (CBV, 3) are limited but it has been shown to be of comparable activity to CBN (1) as an activator of TRPA1 and inhibitor of TRPM8 cation channels but it is a superior modulator of TRPV2, a clinically validated cardiovascular target, but it is less effective on TRPV4. CBV (3) is under consideration as a possible anticancer drug and for other possible uses in combination such as with terpenes (for example see Pollastro, F.; Caprioglio, D.; Marotta, P.; Moriello, A. S.; De Petrocellis, L.; Taglialatela-Scafati, O.; Appendino, G., “Iodine-Promoted Aromatization of p-Menthane-Type Phytocannabinoids”, Journal of Natural Products, 2018, volume 81, pages 630-633; Simón, J. A. P.; González, M. V. B., “Agents for Treating Multiple Myeloma”, US Patent Application 2016/0120874 A1).

The cannabinoid carboxylic acids including cannabinolic acid (CBNA, 2) and cannabivarinic acid (CBVA, 4) currently have limited biological and medical applications. The 4-terpenyl ester of cannabinolic acid (CBNA, 2), isolated amongst other cannabinoid terpene esters from high-potency Mexican Cannabis sativa plants, showed moderate antimicrobial activities against Candida albicans ATCC 90028, Plasmodium falciparum (D6 clone) and Plasmodium falciparum (W2 clone) but showed only low affinity to the CB-1 receptor. Based alone on an in vitro cellular assay, cannabinolic acid (CBNA, 2), amongst other acidic cannabinoids, have been claimed to be of use in increasing the natural resistance of an animal, enhancing cellular resistance, for treating diabetes or atherosclerosis and in reducing the decline in stress response found in ageing. (D'Aniello, E.; Fellous, T.; Iannotti, F. A.; Gentile, A.; Allara, M.; Balestrieri, F.; Gray, R.; Amodeo, P.; Vitale, R. M.; Di Marzo, V., “Identification and characterization of phytocannabinoids as novel dual PPARα/γ agonists by a computational and in vitro experimental approach”, Biochimica et Biophysica Acta General Subjects, 2019, volume 183, pages 586-597; Korthout, H. A. A. J.; Verhoeckx, K. C. M.; Witkamp, R. F.; Doornbos, R. P.; Mei Wang, M., “Medicinal Acidic Cannabinoids: U.S. Pat. No. 7,807,711; Parolaro, D.; Massi, P., Antonio, A.; Francesca Borelli, F.; Aviello, G.; Di Marzo, V.; De Petrocellis, L.; Schiano Moriello, A. S.; Ligresti, A.; Alexandra Ross, R. A.; Ford, L. A.; Anavi-Goffer, S.; Guzman, M.; Velasco, G.; Lorente, M.; Torres, S.; Kikuchi, T.; Guy, G.; Stott, C.; Wright, S.; Sutton, A.; Potter, D.; Etienne De Meijer, E., “Phytocannabinoids in the Treatment of Cancer”, U.S. Pat. No. 8,790,719; Javid, F. A.; Duncan, M.; Stott, C., “Use of Phytocannabinoids in the Treatment of Ovarian Carcinoma”, U.S. Pat. No. 10,098,867; Stott, C.; Duncan, M.; Hill, T., “Active Pharmaceutical Ingredient (API) Comprising Cannabinoids for use in the Treatment of Cancer”, U.S. Pat. No. 9,962,341; Scott, K. A.; Shah, S.; Dalgleish, A. G.; Liu, W. M., “Enhancing the Activity of Cannabidiol and Other Cannabinoids In Vitro Through Modifications to Drug Combinations and Treatment Schedules”, Anticancer Research, 2013, volume 33, pages 4373-4380; Ahmed, S. A.; Ross, S. A.; Slade, D.; Radwan, M. M.; Zulfiqar, F.; ElSohly, M. A., “Cannabinoid Ester Constituents from High-Potency Cannabis sativa”, Journal of Natural Products, 2008 volume 71, pages 536-542; Kariman, A., “Compound and Method for Treating Spasms, Inflammation and Pain”, US Patent Application US 2018/0193399 A1; Korthout, H. A. A. J., “Medical use for Acidic Cannabinoids”, WO Patent Application 2012/144892 A1; Wright, S.; Wilhu, J., Parenteral formulations”, GB Application 2551986).

A vast number of combinations of one, two or three cannabinoids including cannabinolic acid (CBNA, 2) admixed with terpenes have been claimed but their possible uses have not been defined (Levy, K.; Cooper, J. M.; Martin, J. R.; Reid, B. G., “Compositions Purposefully Selected Comprising Purified Cannabinoids and/or Purified Terpenes”, WO Patent Application 2018/160827 A1).

In contrast to these currently limited biomedical applications for the cannabinoid acids 2 and 4, THCA, which is the carboxylic acid precursor of THC, has been widely studied. In a series of preclinical studies, THCA has been shown to be of value in controlling pain including neuropathic pain and fibromyalgia, epilepsy, cancers of the prostate, breast, colon, lung and skin, inflammation including encephalomyelitis as well as autoimmune diseases and to act as an anti-emetic (for examples see Dejana, R. Z.; Folić, M.; Tantoush, Z.; Radovanović, M.; Babić, G.; Janković, S. M., “Investigational cannabinoids in seizure disorders, what have we learned thus far?” Expert Opinion on Investigational Drugs, 2018, volume 27, pages 535-541; Rock, E. M.; Kopstick, L.; Limebeer, C. L.; Parker, L. A., “Tetrahydrocannabinolic acid reduces nausea-induced conditioned gaping in rats and vomiting in Suncus murinus”, British Journal of Pharmacology, 2013, volume 170, pages 641-648; Korthout, H. A. A. J; Verhoeckx, K. C. M.; Witkamp, R. F.; Doornbos, R. P.; Wang, M., “Medicinal Acidic Cannabinoids”, U.S. Pat. No. 7,807,711 B2; Rock, E. M.; Limebeer, C. L.; Navaratnam, R.; Sticht, M. A.; Bonner, N.; Engeland, K.; Downey, R.; Morris, H.; Jackson, M.; Parker, L. A., “A comparison of cannabidiolic acid with other treatments for anticipatory nausea using a rat model of contextually elicited conditioned gaping”, Psychopharmacology, 2014, volume 231, pages 3207-3215; Di Marzo, V.; De Petrocellis, L.; Moriello, A. S., “New use for cannabinoid-containing plant extracts”, G. B. Patent 2,448,535; Parolaro, D.; Massi, P.; Izzo, A. A.; Borelli, F.; Aviello, G.; Di Marzo, V.; De Petrocellis, L.; Moriello, A. S.; Ligresti, A.; Ross, R. A.; Ford, L. A.; Anavi-Goffer, S.; Guzman, M.; Velasco, G.; Lorente, M.; Torres, S.; Kikuchi, T.; Guy, G.; Stott, C.; Wright, S.; Sutton, A.; Potter, D.; De Meijer, E., “Phytocannabinoids in the Treatment of Cancer”, U.S. Pat. No. 8,790,719 B2; Trevor Percival Castor, T. P.; Rosenberry, L. C.; Tyler, T. A.; Student, R. J., “Methods for Making Compositions and Compositions for Treating Pain and Cachexia”, US Patent Application 2008/0103193 AI; Kariman, K., “Compound and Method for Treating Spasms, Inflammation and Pain”, US Patent Application 2018/0193399 AI; Sinai, A.; Turner, Z., “Use of Cannabis to Treat Fibromyalgia, Methods and Compositions Thereof”, WO Patent Application 2016/181394 AI).

If the cannabinoid acids 2 and 4 were to be made available more easily in larger quantities and higher purities, it would be possible to better and more thoroughly examine their uses in medicine either as mono-therapeutic agents or in combination with other cannabinoids or other biologically active compounds. It is germane to note that mixtures of cannabinoids may be more efficacious than single components (the entourage effect). For example, the presence of THCA and other cannabinoids has been shown to enhance the efficacy of THC as an antitumor agent in cell culture and animal models of ER+/PR+, HER2+ and triple-negative breast cancer (for example see Blasco-Benito, S.; Seijo-Vila, M.; Caro-Villalobosa, M.; Tundidor, I.; Andradas, C.; Garcia-Taboada, E.; Wade, J.; Smith, S.; Guzmán, M.; Pérez-Gómez, E.; Gordon, M.; Sánchez, C., “Appraising the “entourage effect”: Antitumor action of a pure cannabinoid versus a botanical drug preparation in preclinical models of breast cancer”, Biochemical Pharmacology, 2018, volume 157, pages 285-293).

The present invention is directed towards overcoming the problems of availability of all the cannabinoids 1 to 4 in high purities by providing efficient/reproducible manufacturing routes for these compounds and providing flexible syntheses of novel cannabinoid analogs, which may be used as active compounds either alone or admixed in combination with known cannabinoids or other drugs in drug formulations for the treatment of pain, multiple sclerosis-related spasticity, nausea, anorexia, epilepsy, Alzheimer's and neurodegenerative diseases, brain injury/concussion/traumatic brain injury, stroke, cancer, reduction of inflammation and immuno-inflammation related diseases, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation among other diseases, and as anti-oxidants.

### SUMMARY OF THE INVENTION

Among the benefits and improvements disclosed herein, other objects and advantages of the disclosed embodiments will become apparent from the following wherein like numerals represent like parts throughout the several figures. Detailed embodiments of cannabinoid compounds, intermediary compounds, and a process for preparation of cannabinoid and cannabimimetic compounds and their intermediaries are disclosed; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “In some embodiments” and “in some embodiments” as used herein do not necessarily refer to the same embodiment(s), though it may. The phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

The purpose of combination or adjuvant therapies herein described are to enhance the efficacy of a drug by the use of a second drug or more drugs or to reduce the dose-limiting toxicities of a drug by the use of a second drug or more drugs.

As used herein, the term “substituted benzyl” means a benzyl ring bearing 1, 2 or 3 independently varied C1-C4 alkyl, C1-C4 alkyloxy, fluoro, chloro, hydroxy, trifluoromethyl, trifluoromethoxy, methylenedioxy, cyano, or methoxymethyl groups at an aromatic ring position or positions or 1 or 2 independently varied C1-C4 alkyl at the benzylic methylene.

If not otherwise defined herein, the term “optionally substituted aryl” means a phenyl ring optionally bearing 1, 2, or 3 independently varied C1-C4 alkyl, C1-C4 alkyloxy, fluoro, or chloro groups.

If not otherwise defined herein, the term “substituted” means optionally substituted at any position with varied C1-C4 alkyl, C1-C4 alkyloxy, fluoro, chloro, hydroxy, trifluoromethyl, trifluoromethoxy, methylenedioxy, cyano, or methoxymethyl groups.

The present invention relates to a process for the preparation of diverse known and novel cannabinoids 5 from the precursors 6 or diastereoisomers or enantiomers of precursors 6 or from the precursors 7 or diastereoisomers or enantiomers of precursors 7 or a mixture of both 6 and 7 or a mixture of any diastereoisomers or enantiomers of precursors 6 and 7 in any ratio via the intermediates 8 and 9 or their diastereoisomers or enantiomers including cannabinol (CBN, 1), cannabinolic acid (CBNA, 2), cannabivarin (CBV, 3), cannabivarinic acid (CBVA, 4) and other naturally occurring related cannabinoids and other synthetic related analogues from simple inexpensive starting materials using a cascade sequence of allylic rearrangement and double aromatization. The present invention also relates to the synthesis of the intermediates 6, 7, 8 and 9 and product cannabinoids 5 (R²≠Me) as racemic modifications or as mixtures of the two enantiomers in non-equal proportions, or as the specific enantiomer shown below or as their enantiomers or diastereoisomers.

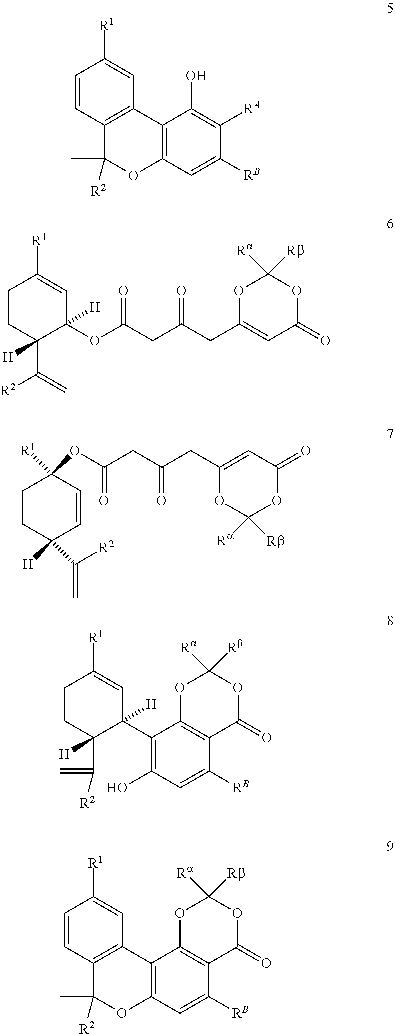

wherein:
R¹ is H, C₁ to C₆ alkyl, (CH₂)ₙ—C₃ to C₆ cycloalkyl, (CH₂)ₘ—OR³;
R² is H, C₁ to C₆ alkyl, (CH₂)ₙ—C₃ to C₆ cycloalkyl, (CH₂)ₘ—OR³;
each n is independently 0, 1 or 2;
each m is independently 1 or 2;
R³ is H, CH₃, CH₂CH₃, CH₂CH₂CH₃ or CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6;
Rα and Rβ are independently C₁ to C₆ alkyl or optionally substituted aryl or Rα and Rβ in combination are (CH₂), (s is 4, 5 or 6) with Rα and Rβ being preferably both methyl.

The synthetic methods are suitable for use on a large scale and for manufacturing purposes. Examples of known cannabinoids that are available using the synthetic routes are cannabinol (CBN, 1), cannabinolic acid (CBNA, 2), cannabivarin (CBV, 3) and cannabivarinic acid (CBVA, 4).

The synthetic methods are also suitable for the synthesis of the novel cannabinoids 5 and these compounds are also part of the invention. The cannabinoids 5 below, which are novel analogs of cannabinol (CBN, 1), cannabinolic acid (CBNA, 2), cannabivarin (CBV, 3) and cannabivarinic acid (CBVA, 4), are also available by the synthetic routes herein described and are part of the invention. These cannabinoids 5 have the formula:

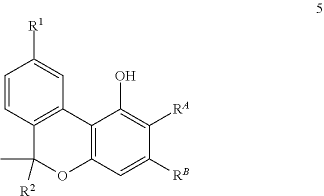

wherein:
R¹ is H, C₁ to C₆ alkyl, (CH₂)ₙ—C₃ to C₆ cycloalkyl, (CH₂)ₘ—OR³;
R² is C₂ to C₆ alkyl, (CH₂)ₙ—C₃ to C₆ cycloalkyl, (CH₂)ₘ—OR³;
each n is independently 0, 1 or 2;
each m is independently 1 or 2;
R³ is H, CH₃, CH₂CH₃, CH₂CH₂CH₃ or CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl with the exclusion of R^{B} being n-propyl or n-pentyl, when R^{A} is H or CO₂H;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R^{F} is C₁ to C₆ alkyl, (CH₂)ᵣ—C₃ to C₆ cycloalkyl;
each r is independently 0, 1, 2, 3, 4, 5 or 6.

The aforementioned novel cannabinoids with the limited formulae 1-4 above may be used as active compounds either alone or admixed in combination with known cannabinoids such as but not limited to Δ⁹-tetrahydrocannabinol (THC), tetrahydrocannabivarin (THCV), cannabidiol (CBD) or cannabidivarin (CBVD) alone or in combination or with other drugs for the treatment of pain, multiple sclerosis-related spasticity, nausea, epilepsy, Alzheimer's brain injury/concussion, cancer, glaucoma and retinal degeneration, disorders of immune-inflammation, lung injury or disease, liver injury or disease, kidney injury or disease, eye injury or disease, amongst other pathologies. In some embodiments, the said novel cannabinoids with the limited formulae 5 above either alone or admixed in combination with known cannabinoids such as but not limited to Δ⁹-tetrahydrocannabinol (THC), tetrahydrocannabivarin (THCV), cannabidiol (CBD), or cannabidivarin (CBDV) alone or in combination or with other drugs are formulated into pharmaceutical compositions in a suitable form for administration to a patient. Such formulations, in addition to the active cannabinoid or cannabinoids or other drugs in a combination therapeutic agent, contain pharmaceutically acceptable diluents and excipients. In the context of this invention, the term excipient encompasses standard excipients well known to a person of ordinary skill in the art (for example see Niazi, S. K., “Handbook of Pharmaceutical Manufacturing Formulations, Compressed Solid Products, 2009, volume 1, pages 67 and 99-169 2^{nd} Edition, Informa Healthcare) but also may include a volatile or mixture of volatile synthetic or isolated monoterpenes from Cannabis sativa and citrus oil. The aforementioned pharmaceutical compositions may be administrated to a patient by enteral, sublingual, intranasal, inhalation, rectal or parenteral drug administration or by other known methods of clinical administration.

### DETAILED DESCRIPTION OF THE INVENTION

### Large Scale-Synthesis of Cannabinol (CBN, 1), Cannabinolic Acid (CBNA, 2), Cannabivarin (CBV, 3), Cannabivarinic Acid (CBVA, 4) and Analogs

The present invention relates to a process for the preparation of diverse known and novel cannabinoids 5 including cannabinol (CBN, 1), cannabinolic acid (CBNA, 2), cannabivarin (CBV, 3) and cannabivarinic acid (CBVA, 4), and other naturally occurring tricyclic cannabinoids from simple inexpensive starting materials using a cascade sequence of allylic rearrangement and double aromatization. The invention also relates to the synthesis of the cannabinoids 5 as racemic modifications or as mixtures of the two enantiomers in non-equal proportions, or as each separate enantiomer shown. The invention includes synthesis of the target cannabinoids as oils or crystalline derivatives, as appropriate, including solvates, hydrates and polymorphs. The process involves the large-scale syntheses of cannabinoids 5:

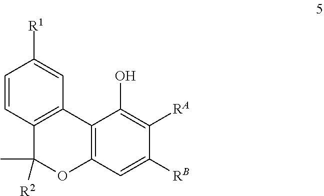

where:
R¹ is H, C₁ to C₆ alkyl, (CH₂)ₙ—C₃ to C₆ cycloalkyl, (CH₂)ₘ—OR³;
R² is H, C₁ to C₆ alkyl, (CH₂)ₙ—C₃ to C₆ cycloalkyl, (CH₂)ₘ—OR³;
each n is independently 0, 1 or 2;
each m is independently 1 or 2;
R³ is H, CH₃, CH₂CH₃, CH₂CH₂CH₃ or CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl, (CH₂)ᵣ—C₃ to C₆ cycloalkyl;
each r is independently 0, 1, 2, 3, 4, 5 or 6;
said process comprising:
treating a first intermediate of the formula 8 or its diastereoisomer or enantiomer, in which any hydroxyl group in R¹ [wherein R¹ is (CH₂)ₘ—OH] or R² [wherein R² is (CH₂)ₘ—OH] is protected, with an oxidant such as iodine in an inert solvent at reflux, preferably by reflux in an aromatic solvent, and most preferably by reflux in toluene to provide the aromatized intermediate 9; or with an oxidant such as iodine in sub-stoichiometric quantities along with another oxidant such as air or oxygen in the presence of a metal catalyst, ligand and an iodide salt in an inert solvent at reflux, preferably by reflux in an aromatic solvent, and most preferably using copper(I) iodide or palladium(II) acetate, sodium iodide and air in the presence of N,N,N′,N′-tetramethyl-1,2-diaminoethane by reflux in toluene; or electrochemically by anodic oxidation in an inert solvent containing a soluble iodide salt and a supporting electrolyte with inert electrodes, preferably in an ether or nitrile solvent with platinum or carbon electrodes, and most preferably in 1,2-dimethoxyethane or acetonitrile with platinum or carbon electrodes using tetra-n-butylammonium iodide or tetraethylammonium iodide and tetra-n-butylammonium hexafluorophosphate or tetraethylammonium hexafluorophosphate;
and secondly hydrolysis of said 9 with optional decarboxylation or by transesterification or by amide formation with optional deprotection as appropriate to provide 5;
or hydrolysis of a first intermediate of the formula 8 or its diastereoisomer or enantiomer, in which any hydroxyl group in R¹ [wherein R¹ is (CH₂)ₘ—OH] or R² [wherein R² is (CH₂)ₘ—OH] with optional decarboxylation or by transesterification or by amide formation with optional deprotection as appropriate to provide a second intermediate of the formula 10,
and secondly treating the second intermediate of the formula 10 or its diastereoisomer or enantiomer, in which any hydroxyl group in R¹ [wherein R¹ is (CH₂)ₘ—OH] or R² [wherein R² is (CH₂)ₘ—OH] is protected, with an oxidant such as iodine in an inert solvent at reflux, preferably by reflux in an aromatic solvent, and most preferably by reflux in toluene to provide the aromatized intermediate 5; or with an oxidant such as iodine in sub-stoichiometric quantities along with another oxidant such as air or oxygen in the presence of a metal catalyst, ligand and an iodide salt in an inert solvent at reflux, preferably by reflux in an aromatic solvent, and most preferably using copper(I) iodide or palladium(II) acetate, sodium iodide and air in the presence of N,N,N′,N′-tetramethyl-1,2-diaminoethane by reflux in toluene; or electrochemically by anodic oxidation in an inert solvent containing a soluble iodide salt and a supporting electrolyte with inert electrodes, preferably in an ether or nitrile solvent with platinum or carbon electrodes, and most preferably in 1,2-dimethoxyethane or acetonitrile with platinum or carbon electrodes using tetra-n-butylammonium iodide or tetraethylammonium iodide and tetra-n-butylammonium hexafluorophosphate or tetraethylammonium hexafluorophosphate.

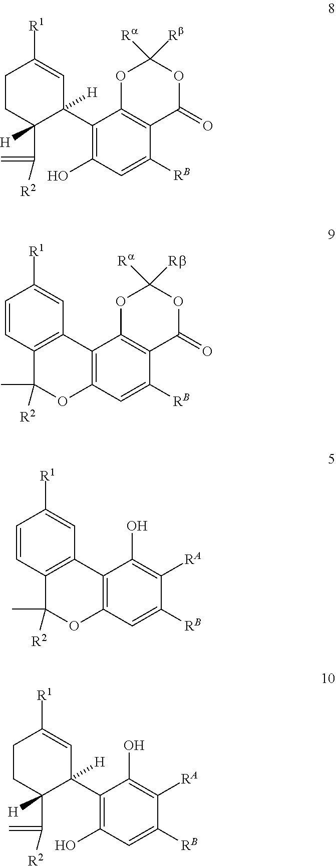

The use of iodine mediated cyclo-etherification and dehydrogenation under either stoichiometric oxidant or electrochemical methods to convert intermediate 8 or its diastereoisomer or enantiomer (R²≠Me) into intermediate 9 has not previously been used in synthesis of any cannabinoids bearing the cyclic dioxinone ring substituent (R^{α}R^{β}C) or in the syntheses of cannabivarin (CBV, 3) and related cannabinoids or related cannabinoid carboxylic acid derivatives but it was reported that cannabidiol (11) and cannabidiolic acid (12) were both converted into CBN (1) by reaction with iodine with in-situ decarboxylation in the reaction from cannabidiolic acid (12). Additionally, condensation of citral (13) with olivetol (14) in the presence of t-butylamine followed by iodine mediated aromatization gave CBN (1) whereas CBC (15) was converted into CBN (1) with iodine and t-butylamine. See (Caprioglio, D.; Mattoteia, D.; Minassi, A.; Pollastro, F.; Lopatriello, A.; Muñoz, E.; Taglialatela-Scafati, O.; Appendino, G., “One-Pot Total Synthesis of Cannabinol via Iodine-Mediated Deconstructive Annulation”, Organic Letters, 2019, volume 21, pages 6122-6125; Pollastro, F.; Caprioglio, D.; Marotta, P.; Schiano Moriello, A.; de Petrocellis, L.; Taglialatela-Scafati, O.; Appendino, G., “Iodine-Promoted Aromatization of p-Menthane-Type Phytocannabinoids”, Journal of Natural Products, 2018, volume 81, pages 630-633). The use of the dioxinone ring system not only permits a simple synthesis of the precursor intermediates 9, it also simplifies the overall reaction and allows for the easy large-scale synthesis of cannabinolic acid (CBNA, 2), cannabivarinic acid (CBVA, 4) and related cannabinoids without decarboxylation during aromatization.

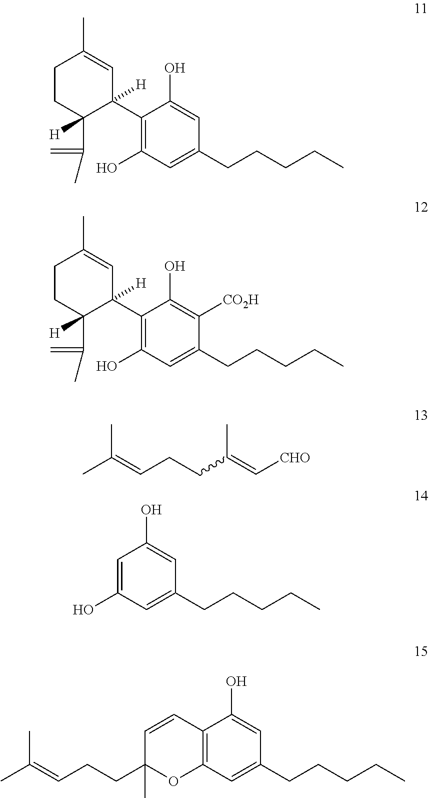

The key intermediates 8 or these large-scale syntheses are prepared by the methods in International Patent Application No. PCT/US2019/47280 from simple inexpensive starting materials using a cascade sequence of allylic rearrangement, aromatization and highly stereoselective and regioselective further cyclization.

Amide formation is carried out by activation of the carboxylic acid for example by formation of the N-hydroxysuccinimide ester and coupling with the corresponding amine, for example see Goto (Y. Goto, Y. Shima, S. Morimoto, Y. Shoyama, H. Murakami, A. Kusai and K. Nojima, “Determination of tetrahydrocannabinolic acid-carrier protein conjugate by matrix-assisted laser desorption/ionization mass spectrometry and antibody formation”, Journal of Mass Spectrometry, 1994, volume 29, pages 668-671). Alternative amide coupling reagents include but are not limited to dicyclohexyl carbodiimide (DCC), di-iso-propyl carbodiimide (DIC), O-(7-azabenzotriazol-1-yl)-1,1,3,3-tetramethyluronium hexafluorophosphate (HATU), O-(benzotriazol-1-yl)-1,1,3,3-tetramethyluronium hexafluorophosphate (HBTU) and bromotri(pyrrolidino)phosphonium hexafluorophosphate (PyBrop) (E. Valeur and M. Bradley, “Amide bond formation: beyond the myth of coupling reagents”, Chemical Society Reviews, 2009, volume 38, pages 606-631).

The present invention also relates to a related process for the preparation of diverse known and novel cannabinoids 5 including cannabinol (CBN, 1), cannabinolic acid (CBNA, 2), cannabivarin (CBV, 3) and cannabivarinic acid (CBVA, 4) and other naturally occurring tricyclic cannabinoids and other synthetic tricyclic analogues from the intermediates 9 or their diastereoisomers or enantiomers and subsequent transformation into the cannabinoids 5.

The iodine mediated dehydrogenation reaction is suitable for the synthesis of novel cannabinoids 5 and these compounds are also part of the invention. The invention includes the synthesis of the target cannabinoids as oils or crystalline derivatives, as appropriate, including solvates, hydrates and polymorphs. These novel cannabinoids 5 have the formula:

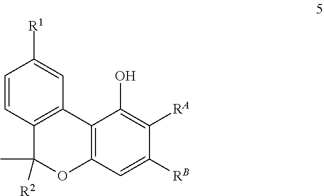

wherein:
R¹ is C₂ to C₆ alkyl, (CH₂)ₙ—C₃ to C₆ cycloalkyl, (CH₂)ₘ—OR³;
R² is H, C₁ to C₆ alkyl, (CH₂)ₙ—C₃ to C₆ cycloalkyl, (CH₂)ₘ—OR³;
each n is independently 0, 1 or 2;
each m is independently 1 or 2;
R³ is H, CH₃, CH₂CH₃, CH₂CH₂CH₃ or CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;

R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl, (CH₂)ᵣ—C₃ to C₆ cycloalkyl;
each r is independently 0, 1, 2, 3, 4, 5 or 6;
   with the exception of cannabinol (CBN, 1), cannabinolic acid (CBNA, 2), cannabivarin (CBV, 3) and cannabivarinic acid (CBVA, 4), cannabinoids 5 [R¹═R²=Me and R^{A}═H with R^{B}═C(Me)₂-n-C₆H₁₃, R^{B}═C(Me)₂-n-C₃H₇, R^{B}═C(Me)₂-CH₂CH₂CH₂-iso-Pr, R^{B}=n-C₂H₅, R^{B}=n-C₃H₇, R^{B}=n-C₄H₉, R^{B}=n-C₅H₉, R^{B}═CH₂CH(CH₃)-n-C₅H₁₁, R^{B}═CH(CH₃)CH(CH₃)-n-C₄H₉, R^{B}═CH(C₂H₅)-n-C₆H₁₃, R^{B}═CH(CH₃)CH(CH₃)-n-C₅H₁₁, R^{B}═CH(CH₃)CH(CH₃)-n-C₆H₁₃, R^{B}═CH(OH)-n-C₄H₉], 5 [R¹═R²=Me and R^{A}═H with R^{B}=Me, R^{B}═C(Me)₂-n-C₃H₇, R^{B}=n-C₇H₁₅, R^{B}=n-C₆H₁₃, R^{B}=n-C₄H₉, R^{B}═CH(Et)₂], 5 [R¹═R²=Me and R^{A}═H with R^{B}═CH(OH)-n-C₄H₉, R^{B}═CH₂CH₂CH₂CH₂CH₂OH, R^{B}═CH₂CH(OH)-n-C₃H₇, R^{B}═CH₂CH₂CH(OH)CH₂Me, R^{B}═CH₂CH₂CH₂CH(OH)Me], 5 [R¹═CH₂OH, R²=Me and R^{A}═H with R^{B}═C(Me)₂-n-C₆H₁₃, R^{B}═CH(OH)-n-C₄H₉, R^{B}═CH₂CH(OH)-n-C₃H₇, R^{B}═CH₂CH₂CH(OH)-n-C₂H₅, R^{B}=n-C₅H₁₁, R^{B}=n-C₅H₁₁, R^{B}═(CH₂)₃CH(OH)CH₃, 5 [R¹═R²=Me and R^{A}═CO₂Me, R^{B}=n-C₃H₇, R^{B}=n-C₅H₉], 5 [R¹═CH₂CH₃, R²=Me and R^{A}═H, R^{B}═CH(CH₃)CH(CH₃)-n-C₅H₁₁ and 5 [R¹=n-C₄H₉, R²=Me and R^{A}═H, R^{B}═H.

The dioxinone resorcylate derivatives 9 below, which are intermediates for the synthesis of cannabinoids, are also available by the synthetic routes herein described and are part of the invention. These novel dioxinone derivatives 9 have the formula:

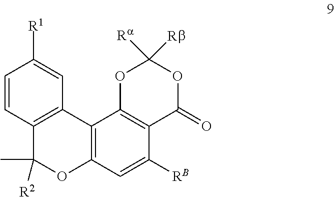

wherein:
R¹ is C₂ to C₆ alkyl, (CH₂)ₙ—C₃ to C₆ cycloalkyl, (CH₂)ₘ—OR³;
R² is H, C₁ to C₆ alkyl, (CH₂)ₙ—C₃ to C₆ cycloalkyl, (CH₂)ₘ—OR³;
each n is independently 0, 1 or 2;
each m is independently 1 or 2;
R³ is H, CH₃, CH₂CH₃, CH₂CH₂CH₃ or CH(CH₃)₂;
R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is C₁ to C₆ alkyl, (CH₂)_{q}—C₃ to C₆ cycloalkyl, allyl, benzyl, substituted benzyl or 2-phenylethyl;
q is 0, 1, 2, 3, 4, 5 or 6;
R^{D} is C₁ to C₆ alkyl, (CH₂)ᵣ—C₃ to C₆ cycloalkyl, C₃ to C₆ cycloalkyl, allyl, benzyl, substituted benzyl or 2-phenylethyl; R^{E} is C₁ to C₆ alkyl, (CH₂)ᵣ—C₃ to C₆ cycloalkyl, allyl, benzyl, substituted benzyl or 2-phenylethyl; or NR^{D}R^{E} is azetidinyl, pyrrolidinyl, morpholinyl or piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl, (CH₂)ᵣ—C₃ to C₆ cycloalkyl;
each r is independently 0, 1, 2, 3, 4, 5 or 6; and
each of Rα and Rβ is independently C₁ to C₆ alkyl or optionally substituted aryl, or Rα and Rβ in combination are (CH₂)ₛ, wherein s is 4, 5 or 6, with Rα and Rβ being preferably both methyl.

### Example

### Cannabinol (CBN, 1)

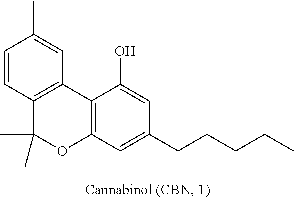 [SMILES: CCCCCc1cc(O)c2c(c1)OC(C)(C)c1ccc(C)cc1-2]

Iodine (254 mg, 1 mmol) was added in one portion to a solution of cannabidiol 10 (R¹═R²=Me; R^{A}═H, R^{B}=n-C₅H₁₁) (155 mg, 0.5 mmol) in toluene (10 mL). The solution was heated at reflux for three hours. At this stage, the reaction was complete by TLC analysis. The solution was cooled to room temperature, diluted with Et₂O (10 mL), and washed with saturated aqueous sodium thiosulfate (2×10 mL). The organic phase was dried over MgSO₄, filtered, and concentrated under reduced pressure to afford an oily residue. Purification of the crude product by flash column chromatography with silica (Et₂O:pentane 1:20 to 2:20) gave cannabinol (1) (80 mg, 0.26 mmol, 52%) as a yellow oil, as confirmed by the following analyses:

¹H NMR (400 MHz, CDCl₃) δ 8.17 (d, J=1.5 Hz, 1H), 7.15 (d, J=7.8 Hz, 1H), 7.09-7.04 (d, 1H), 6.44 (d, J=1.6 Hz, 1H), 6.29 (d, J=1.6 Hz, 1H), 5.26 (s, 1H), 2.52-2.47 (m, 2H), 2.39 (d, J=0.7 Hz, 3H), 1.64-1.61 (m, 1H), 1.60 (d, J=1.5 Hz, 9H), 1.42-1.29 (m, 6H), 0.95-0.84 (m, 3H).

¹³C NMR (101 MHz, CDCl₃) δ 154.7, 153.2, 144.7, 137.0, 127.7, 127.7, 126.5, 122.8, 110.9, 110.0, 108.8, 35.8, 31.6, 30.6, 27.2, 22.7, 21.7, 14.2.

IR (neat) 3384, 2954, 2925, 1620, 1581, 1282, 1045, 731.

HRMS (ES+) m/z calculated for C₂₁H₂₇O₂ [M+H]⁺ 311.2006, found 311.1992.

1. A process for the preparation of a product compound of the formula 5: 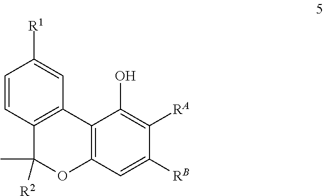
wherein:
R¹ is selected from the group consisting of H, C₁ to C₆ alkyl, (CH₂)ₙ—C₃ to C₆ cycloalkyl, and (CH₂)ₘ—OR³;
R² is selected from the group consisting of H, C₁ to C₆ alkyl, (CH₂)ₙ—C₃ to C₆ cycloalkyl, and (CH₂)ₘ—OR³;
each n is independently 0, 1 or 2;
each m is independently 1 or 2;
R³ is selected from the group consisting of H, CH₃, CH₂CH₃, CH₂CH₂CH₃ and CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;
R^{B} is selected from the group consisting of H or C₁ to C₂ alkyl, linear or branched C₃ to C₁₀ alkyl or double branched C₄ to C₁₀ alkyl, in each case optionally substituted by one or two hydroxyl groups or optionally substituted by one or more fluoro-groups, (CH₂)ₒ—C₃ to C₆ cycloalkyl, (CH₂)ₚ—OR^{F}, or is C₃ to C₆ cycloalkyl optionally substituted by a C₁ to C₈ alkyl;
is 0, 1, 2, 3, 4, 5 or 6;
p is 1, 2, 3, 4, 5 or 6;
R^{C} is selected from the group consisting of C₁ to C₆ alkyl, (CH₂)_{q}—C₃ to C₆ cycloalkyl, allyl, benzyl, substituted benzyl and 2-phenylethyl, wherein
q is 0, 1, 2, 3, 4, 5 or 6;
R^{D} is selected from the group consisting of C₁ to C₆ alkyl, (CH₂)ᵣ—C₃ to C₆ cycloalkyl, allyl, benzyl, substituted benzyl and 2-phenylethyl; and
R^{E} is selected from the group consisting of C₁ to C₆ alkyl, (CH₂)ᵣ—C₃ to C₆ cycloalkyl, allyl, benzyl, substituted benzyl and 2-phenylethyl; or
NR^{D}R^{E} is selected from the group consisting of azetidinyl, pyrrolidinyl, morpholinyl and piperidinyl, each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl or (CH₂)ᵣ—C₃ to C₆ cycloalkyl;
each r is independently 0, 1, 2, 3, 4, 5 or 6;
said process comprising:
providing a first intermediate of the formula 8 or its diastereoisomer or enantiomer: 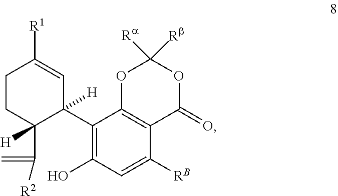
wherein each of Rα and Rβ is independently C₁ to C₆ alkyl or optionally substituted aryl, or
Rα and Rβ in combination are (CH₂)ₛ and s is 4, 5 or 6, and
in which any hydroxyl group if present in R¹ or R² is protected,
treating the first intermediate of the formula 8 with iodine in an inert solvent at reflux; or iodine in sub-stoichiometric quantities along with another oxidant in the presence of a metal catalyst, ligand and an iodide salt in an inert solvent at reflux; or
electrochemically by anodic oxidation in an inert solvent including a soluble iodide salt and a supporting electrolyte with inert electrodes;
to provide the aromatized intermediate of the formula 9: 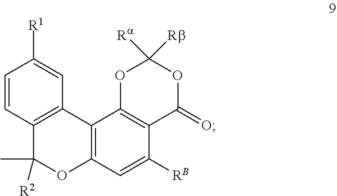
and hydrolyzing the aromatized intermediate 9 with optional decarboxylation or by transesterification or by amide formation with optional deprotection to provide the compound of formula 5; or
hydrolyzing the first intermediate of the formula 8 or its diastereoisomer or enantiomer, in which any hydroxyl group if present in R¹ or R² is protected,
with optional decarboxylation or by transesterification or by amide formation with optional deprotection to provide a second intermediate of the formula 10: 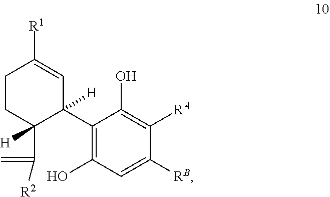
or its diastereoisomer or enantiomer, and
treating the second intermediate of the formula 10 or its diastereoisomer or enantiomer, in which any hydroxyl group if present in R¹ or R² is protected,
with iodine in an inert solvent at reflux; or with iodine in sub-stoichiometric quantities along with another oxidant such as air or oxygen in the presence of a metal catalyst, ligand and an iodide salt in an inert solvent at reflux; or
electrochemically by anodic oxidation in an inert solvent including a soluble iodide salt and a supporting electrolyte with inert electrodes.

2. The process according to claim 1, wherein the step of treating the first intermediate is carried out using iodine by reflux in an aromatic solvent.

3. The process according to claim 1, wherein the step of treating the first intermediate is carried out using iodine by reflux in toluene.

4. The process according to claim 1, wherein the step of treating the first intermediate is carried out using iodine in sub-stoichiometric quantities along with air as oxidant in the presence of a metal catalyst, ligand and an iodide salt in an inert solvent at reflux.

5. The process according to claim 1, wherein the step of treating the first intermediate is carried out using iodine in sub-stoichiometric quantities along with air as oxidant in the presence of copper(I) iodide, a ligand and sodium iodide in an aromatic solvent at reflux.

6. The process according to claim 1, wherein the step of treating the first intermediate is carried out using iodine in sub-stoichiometric quantities along with air as oxidant in the presence of copper(I) iodide, N,N,N′,N′-tetramethyl-1,2-diaminoethane and sodium iodide in toluene at reflux.

7. The process according to claim 1, wherein the step of treating the first intermediate is carried out using iodine in sub-stoichiometric quantities along with air as oxidant in the presence of palladium(II) iodide, a ligand and sodium iodide in an aromatic solvent at reflux.

8. The process according to claim 1, wherein the step of treating the first intermediate is carried out using iodine in sub-stoichiometric quantities along with air as oxidant in the presence of palladium(II) iodide, N,N,N′,N′-tetramethyl-1,2-diaminoethane and sodium iodide in toluene at reflux.

9. The process according to claim 1, wherein the step of treating the first intermediate is carried out electrochemically by anodic oxidation in an inert solvent containing a soluble iodide salt and a supporting electrolyte and with inert electrodes.

10. The process according to claim 1, wherein the step of treating the first intermediate is carried out electrochemically with platinum or carbon electrodes by anodic oxidation in an ether or nitrile solvent containing a soluble iodide salt and a supporting electrolyte.

11. The process according to claim 1, wherein the step of treating the first intermediate is carried out electrochemically with platinum or carbon electrodes by anodic oxidation in 1,2-dimethoxyethane or acetonitrile containing a tetra-n-butylammonium iodide or tetraethylammonium iodide and tetra-n-butylammonium hexafluorophosphate or tetraethylammonium hexafluorophosphate.

12. A compound of formula 5: 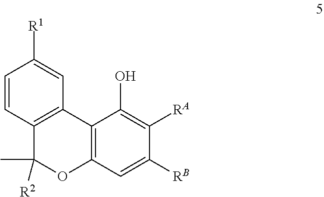
wherein:
R¹ is selected from the group consisting of C₂ to C₆ alkyl, (CH₂)ₙ—C₃ to C₆ cycloalkyl, (CH₂)ₘ—OR³;
R² is selected from the group consisting of H, C₁ to C₆ alkyl, (CH₂)ₙ—C₃ to C₆ cycloalkyl, (CH₂)ₘ—OR³;
each n is independently 0, 1 or 2;
each m is independently 1 or 2;
R³ is selected from the group consisting of H, CH₃, CH₂CH₃, CH₂CH₂CH₃, and CH(CH₃)₂;
R^{A} is H, CO₂H and its pharmaceutically acceptable salts or pharmaceutically acceptable co-crystals, CO₂R^{C} and its pharmaceutically acceptable co-crystals, CONHR^{D} and its pharmaceutically acceptable co-crystals, CONR^{D}R^{E} and its pharmaceutically acceptable co-crystals;
R^{B} is selected from the group consisting of H or C₁ to C₂ alkyl, linear or branched C₃ to C₁₀ alkyl or double branched C₄ to C₁₀ alkyl, in each case optionally substituted by one or two hydroxyl groups or optionally substituted by one or more fluoro-groups, (CH₂)ₒ—C₃ to C₆ cycloalkyl, (CH₂)ₚ—OR^{F}, or is C₃ to C₆ cycloalkyl optionally substituted by a C₁ to C₈ alkyl;
o is 0, 1, 2, 3, 4, 5 or 6;
p is 1, 2, 3, 4, 5 or 6;
R^{C} is selected from the group consisting of C₁ to C₆ alkyl, (CH₂)_{q}—C₃ to C₆ cycloalkyl, allyl, benzyl, substituted benzyl and 2-phenylethyl;
q is 0, 1, 2, 3, 4, 5 or 6;
R^{D} is selected from the group consisting of C₁ to C₆ alkyl, (CH₂)ᵣ—C₃ to C₆ cycloalkyl, C₃ to C₆ cycloalkyl, allyl, benzyl, substituted benzyl or 2-phenylethyl; and
R^{E} is C₁ to C₆ alkyl, (CH₂)ᵣ—C₃ to C₆ cycloalkyl, allyl, benzyl, substituted benzyl or 2-phenylethyl; or
NR^{D}R^{E} is selected from the group consisting of azetidinyl, pyrrolidinyl, morpholinyl and piperidinyl each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl or (CH₂)ᵣ—C₃ to C₆ cycloalkyl; and
each r is independently 0, 1, 2, 3, 4, 5 or 6;
with the exception of cannabinol (CBN, 1), cannabinolic acid (CBNA, 2), cannabivarin (CBV, 3) and cannabivarinic acid (CBVA, 4),
cannabinoids of the formula 5, wherein R¹ and R² are each Me, R^{A} is H, and R^{B} is C(Me)₂-n-C₆H₁₃, C(Me)₂-n-C₃H₇, C(Me)₂-CH₂CH₂CH₂-iso-Pr,
cannabinoids of the formula 5, wherein R¹ and R² are each Me, R^{A} is H, and R^{B} is Me, C(Me)₂-n-C₃H₇, C(Me)₂-CH₂CH₂CH₂-iso-Pr, n-C₇H₁₅, n-C₆H₁₃, n-C₄H₉, CH(Et)₂,
cannabinoids of the formula 5, wherein R¹ and R² are each Me, R^{A} is H, and R^{B} is CH(OH)-n-C₄H₉, CH₂CH₂CH₂CH₂CH₂OH, CH₂CH(OH)n-C₃H₇, CH₂CH₂CH(OH)CH₂Me, CH₂CH₂CH₂CH(OH)Me,
cannabinoids of the formula 5, wherein R¹ is CH₂OH, R² is Me, R^{A} is H, and R^{B} is C(Me)₂-n-C₆H₁₃, CH(OH)-n-C₄H₉, or CH₂CH(OH)-n-C₃H₇],
cannabinoids of the formula 5, wherein R¹ and R² are each Me, R^{A} is CO₂H, and R^{B} is n-C₃H₇], and
cannabinoids of the formula 5, wherein R¹ and R² are each Me, R^{A} is CO₂Me, and R^{B} is n-C₃H₇.

13. An intermediate compound of formula 9: 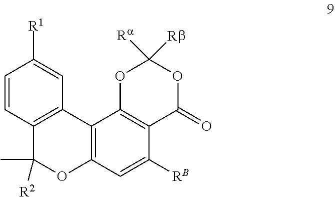
wherein:
R¹ is selected from the group consisting of C₂ to C₆ alkyl, (CH₂)ₙ—C₃ to C₆ cycloalkyl, and (CH₂)ₘ—OR³;
R² is selected from the group consisting of H, C₁ to C₆ alkyl, (CH₂)ₙ—C₃ to C₆ cycloalkyl, and (CH₂)ₘ—OR³;
each n is independently 0, 1 or 2;
each m is independently 1 or 2;
R³ is selected from the group consisting of H, CH₃, CH₂CH₃, CH₂CH₂CH₃ and CH(CH₃)₂;
R^{B} is selected from the group consisting of H or C₁ to C₂ alkyl, linear or branched C₃ to C₁₀ alkyl or double branched C₄ to C₁₀ alkyl, in each case optionally substituted by one or two hydroxyl groups or optionally substituted by one or more fluoro-groups, (CH₂)ₒ—C₃ to C₆ cycloalkyl, (CH₂)ₚ—OR^{F}, or is C₃ to C₆ cycloalkyl optionally substituted by a C₁ to C₈ alkyl;
is 0, 1, 2, 3, 4, 5 or 6;
p is 1, 2, 3, 4, 5 or 6;
R^{C} is selected from the group consisting of C₁ to C₆ alkyl, (CH₂)_{q}—C₃ to C₆ cycloalkyl, allyl, benzyl, substituted benzyl and 2-phenylethyl;
q is 0, 1, 2, 3, 4, 5 or 6;
R^{D} is selected from the group consisting of C₁ to C₆ alkyl, (CH₂)ᵣ—C₃ to C₆ cycloalkyl, C₃ to C₆ cycloalkyl, allyl, benzyl, substituted benzyl and 2-phenylethyl; and
R^{E} is selected from the group consisting of C₁ to C₆ alkyl, (CH₂)ᵣ—C₃ to C₆ cycloalkyl, allyl, benzyl, substituted benzyl and 2-phenylethyl; or
NR^{D}R^{E} is selected from the group consisting of azetidinyl, pyrrolidinyl, morpholinyl and piperidinyl, each optionally substituted by one or two hydroxyl groups or hydroxymethyl groups with the exception that the hydroxyl groups cannot be on the carbon bearing the heterocyclic ring nitrogen or the heterocyclic ring oxygen with morpholine;
R_{F} is C₁ to C₆ alkyl or (CH₂)ᵣ—C₃ to C₆ cycloalkyl; and
each r is independently 0, 1, 2, 3, 4, 5 or 6, and
wherein each of Rα and Rβ is independently C₁ to C₆ alkyl or optionally substituted aryl, or
Rα and Rβ in combination are (CH₂)ₛ and s is 4, 5 or 6.

14-17. (canceled)

18. The process according to claim 1, wherein the another oxidant is air or oxygen.
